(19) United States
(12) Patent Application Publication
(10) Pub. No.: US 2002/0114794 A1
(43) Pub. Date: Aug. 22, 2002

(54) Staphylococcus aureus culture and preparation thereof

(76) Inventor: Juyu Chen, Shenyang (CN)

Correspondence Address: FULBRIGHT & JAWORSKI, LLP, 666 FIFTH AVE, NEW YORK, NY, 10103-3198, US

(21) Appl. No.: 10/074,166

(22) Filed: Feb. 12, 2002

(30) Foreign Application Priority Data
Feb. 16, 2001 (CN) 01 1 04103

(51) Int. Cl. A61K 45/00; C12N 1/20

(52) U.S. Cl. 424/93.42; 435/252.1

(57) ABSTRACT

The invention is related to a culture of Staphylococcus aureus having anti-tumor functions and preparation thereof.

### FIELD OF THE INVENTION

The invention is related to a culture of Staphylococcus aureus having anti-tumor functions and preparation thereof.

### BACKGROUND OF THE INVENTION

It is well known to the person skilled in the arts that the culture of Staphylococcus aureus contains anti-tumor elements, of which the effective elements contain staphylococcal enterotoxin, (SE). Currently SE is regarded as a super-antigen (SAg) with very strong bioactivity. It is reported in the available documents that said super-antigen includes bacteria, viruses and parasites and the like. The meaning, functions and active mechanisms of super-antigens are completely different from the common antigens available now. The super-antigens are of a type of protein from complicated resources and are needed in a tiny quantity in immune response, for example, it can be calculated in ngM. The super-antigens stimulate T cells to reproduce massively with a very special mechanism to generate a large quantity of cell factors. Their cytotoxicity include: (1) to activate T lymphocytes directly to kill target cells, (2) to activate NK cells with cell factors, (3) to kill tumor cells with super-antigen dependent cell-mediated cytotoxicity (SDCC).

In recent years, after super-antigens were proved to have anti-tumor capabilities, McAb-SEA conjugate was produced by several chemical methods, and fusion protein was made with recombination technologies. The fusion protein was put into phase I clinical trial in 1997, and the achieved results are as follows: it is safe to use the dosage of 1.5 ng/kg once, but patients suffer from comparatively strong general reaction, and most of the patients suffer from obvious reduction of leucocyte, and even more reduction in platelet numbers.

### BRIEF SUMMARY OF THE INVENTION

In order to overcome the shortcomings of the prior arts, the object of the invention is to provide a new kind of strain of Staphylococcus aureus.

The another object of the invention is to provide a staphylococcus aureus culture and culture method, as well as the culture medium adopted in the cultivation.

The further object of the invention is to provide the anti-tumor functions use of the above-mentioned Staphylococcus aureus culture of the present invention.

The invention adopts a new culture medium to cultivate and prepare a culture for Staphylococcus aureus. The culture has a function of anti-tumor. The culture medium adopted in the invention is simple in formula so that the quantity of raw materials is reduced. And disinfection treatment is not necessary before the filtrate is used for therapy purposes. The stability of the enzyme produced by the Staphylococcus aures is promoted. After the therapy with the culture, the patients' leucocyte number is increased dramatically, and anti-tumor effect is promoted.

A strain of Staphylococcus aures used in the invention has been deposited in China General Microbiological Culture Collection Center (CGMCC) on Sep. 14, 2000, with a deposit No. CGMCC0485.

The strain CGMCC No. 0485 was cultivated with nitrosoguanidine mutagenesis from strain CGMCC No. 0165, which was sieved from over 100 Staphylococcus aureses separated from sample of hospitals' clinical bacteria laboratory.

Strain CGMCC No. 0485's features are given below:

Morphological Features

The standard Staphylococcus aures and Staphylococcus aures to be examined were amplified by 20,000 times (embedding) and 60,000 times (ultrathin sectioning) with transmission electron microscope, and it can be seen that bacteria are in lines of single balls from 0.5 to 1.0 μm and aligned up in pairs. It showed more than one splitting, and irregular blocks are formed up with no flagellum, no motion, no membrane, and no spore. When viewed with electron microscope (ultrathin sectioning), cell wall, cell membrane and chromatin are all visible. The bacteria of the present strain of different growth phases are different in size and structure. But there exist no obvious differences between the standard Staphylococcus aures and the strain of Staphylococcu aures to be examined.

Cultivation Features

In blood plate culture, the strain CGMCC No. 0485 is in a circular shape with a smooth surface, tidy edge, it appears nontransparent and presents golden color. It has a large and transparent hemolytic ring. Its diameter reaches 1.52˜2 mm after 24 hours of cultivation at 35° C. In liquid medium, the grower is turbid at the beginning and becomes clear afterwards with thin and floating deposits. A circular membrane usually occurs after 2˜3 days of cultivation. Liquid culture is slightly low in turbidity and bacteria concentration under the same conditions.

Staining Features

The standard Staphylococcus aures and the bacteria to be examined were stained with standard Gram staining methods, and observed with microscope. The experiment results showed that both the standard Staphylococcus aures and the said strain to be examined were Gram positive bacteria.

The standard Staphylococcus aures and the bacteria to be examined were stained with membrane staining methods, and observed with microscope. The experiment results showed that both bacteria of the standard Staphylococcus aures and the strain to be examined were negative.

The standard Staphylococcus aures and the bacteria to be examined were stained with spore staining methods, and observed with microscope. The experiment results showed that both the standard Staphylococcus aures and the present bacteria to be examined were negative.

Physiological and Biochemical Features

Plasma coagulase is positive, and enzyme-generating capability in the culture medium is very strong.

Glucose and mannite ferment experiment: acid-producing capability is weak.

Pyrogenicity qualification rate of the final product after degerming of the culture is high, which is higher than 95%.

Starches hydrolysis experiment: both the standard Staphylococcus aures and bacteria of the present invention are negative.

Catalase experiment: both the standard Staphylococcus aures and bacteria of the present invention are positive.

The process of producing a Staphylococcus aures culture of the invention includes the steps of:

(1) putting the raw material of pig heart into 1˜2 times of water, and shattering and filtrating it to obtain the first filtrate and the second filter residue;

(2) putting 1˜2 times of water into the filter residue and immersing and filtrating the same at 90˜95° C. to obtain the second filtrate and second filter residue; mixing the first and second filtrate to obtain the third filtrate;

(3) putting 0.025˜0.5% of peptone and 0.3˜0.9% sodium chloride into the third filtrate based on the total liquid weight,, and adding 0.5% active carbon, adjusting its pH value to about 7.2 to obtain a culture medium;

(4) reactivating Staphylococcus aures, and preparing seed liquid after bacteria number is increased, inoculating the a culture medium with a rate of 0.02%˜0.2% ml % after bacteria concentration reaches 10⁵-10⁷;

(5) fermenting inoculated medium at about 35° C. for 15˜20 hours to get a culture;

(6) removing the bacteria , filtrating said culture, and adjusting its osmotic pressure to make it isotonic, and pH value to 6.8˜7.4, so as to obtain the final product.

So, in the present invention, the said culture medium includes 0.5˜10 ml of pig heart filtrate, 0.025˜0.5% of peptone and 0.3˜0.9% sodium chloride based on the total medium weight.

The product of the invention can be processed directly and made into injection preparations. Its dosage for patients suffering tumors is one ampoule of 2 ml each day.

The above-mentioned strain of Staphylococcus aures has been deposited in China General Microbiological Culture Collection Center (CGMCC) since Sep. 14, 2000 with the number of CGMCC 0485.

It shortens the fermenting time by ⅕ to adopt the present Staphylococcus aures, and shortens culture procedure for the seed liquid by ⅔. It reduces the consumption quantity of the raw materials, and is simple in techniques and easy in control. It can dramatically promote the product's quality while reducing the cost. Besides, the enzyme producing capability is also increased after mutagenesis. The Staphylococcus aures requirements on culture conditions are comparatively low, and pyrogenicity qualification rate of the final product is dramatically increased while the side effects are decreased.

### DETAILED DESCRIPTION OF THE INVENTION

Embodiments are made as follows for better understanding of the present invention.

### EXAMPLE 1

100 grams of pig heart was taken and cleaned out with water, following by grinding with meat grinder (model TJL12-4 made by Guangdong Panyu Meat Food Mechanism Factory) to get chopped meat. The said chopped meat was mixed with 150 kg injection and kept soaking for one hour at 90° C. Then the pig heart filtrate was obtained by filtration. Further 100 kg of injection water was put into the filtrated filter residue, and kept soaking for one hour at 90° C., and then another filtrate was gotten while discarding the filter residue. All obtained pig heart filtrate were combined together to have a filtrate mixture. 2,000 ml of the combined filtrate mixture was added into 100 grams of peptone and 1,200 grams of sodium chloride, after heating it was kept stirring and boiled so that all added materials were dissolved completely. Then clean water was further added into it and kept its pH value being 8.5. It stood overnight at 4° C. 10 grams of active carbon were put into the liquid and adjusting its pH value from 8.5 to about 7.2 gradually.

The above filtrate was adjusted into isotonic liquid and filled into sealing bottles of 500 ml, and sterilizing for 20 minutes under 0.1 Mpa to obtain 400 kg of culture medium, of which the total volume is about 400,000 ml. The Staphylococcus aure No.CGMCC 0485 was reactivated and kept for 24 hours at 35° C. Then it was leaved in blood Petri dish for 8 hours at 35° C. to increase amount of bacteria to obtain a seed liquid. The bacterium concentration in the seed liquid is 10⁷.

According to the present invention, culture medium was mixed in a stainless steel container. After sterilization and filtration, it was filled into a ferment tank. It was preheated to 35° C. and inoculated with the quantity of 0.2% ml and fermented for 16 hours at 35° C. in order to have a culture. The culture was filtrated. After checked and tested, it packed into injection preparations.

All the cultures obtained with fermentation should pass the tests of pyrogenicity, enzyme activity and allergic experiment.

### EXAMPLE 2

100 kg of pig heart was taken and cleaned out with water, following by grinding with meat grinder (model TJL12-4 made by Guangdong Panyu Meat Food Mechanism Factory) to get chopped meat. The said chopped meat was mixed with 150 kg injection and kept soaking for one hour at 90° C. Then the pig heart filtrate was obtained by filtration.

Further 100 kg of injection water was put into the filtrated filter residue, and kept soaking for one hour at 90° C., and then the filtrate was gotten while discarding the filter residue. All obtained pig heart filtrate were combined together to have a filtrate mixture. 4,000 ml of the combined filtrate mixture was added into 2000 grams of peptone and 3,600 grams of sodium chloride, after heating it was kept stirring and boiled so that all added materials were dissolved completely. Then clean water was further added into it and kept its pH value being about 8.5. It stood overnight at 4° C. 200 grams of active carbon were put into the liquid and adjusting its pH value from 8.0 to about 7.0 gradually.

The above filtrate was adjusted into isotonic liquid and filled into sealing bottles of 500 ml and sterilizing for 20 minutes under 0.1 Mpa to obtain 400 kg of culture medium, of which the total volume is about 400,000 ml. The Staphylococcus aure No. CGMCC 0485 was reactivated and kept for 24 hours at 35° C. Then it was leaved in blood Petri dish for 8 hours at 35° C. to increase amount of bacteria to obtain a seed liquid. The bacterium concentration in the seed liquid is 10⁷.

According to the present invention, culture medium was mixed in a stainless steel container. After sterilization and filtration, it was filled into a ferment tank. It was preheated to 35° C. and inoculated with the quantity of 0.2% ml and fermented for 16 hours at 35° C. in order to have a culture broth. The culture broth was filtrated. After checked and tested, it packed into injection preparations.

All the cultures obtained with fermentation should pass the tests of pyrogenicity, enzyme activity and allergic experiment.

### Experimental Example 1

Experiment Of Anti-Leucopenia Caused by Chemotherapy

The culture obtained in the example 1 was used in China-Japan Friendship Hospital, Beijing for clinical experiment of anti-leucopenia caused by chemotherapy. 20 cases of cancer, mainly lung cancer (over 60%) accepting chemotherapy were selected. It was proved pathologically and cytologically in the experiment that intramuscular injection of the present culture caused no affects on patients' livers and kidneys before and after the therapy. But it evidently prevented leucocyte from decrease caused by chemotherapy (p<0.05 and P<0.01). Its effective rate and notable effective rate in the period of therapy reached 90% and 55% while the contrast effective rate and notable rate were respectively 15% and 5%.

As a conclusion, the culture of the present invention has the capability of anti-leucopenia caused by chemotherapy, and is able to prevent or alleviate leucopenia, shorten the time of leucopenia and accelerate the recovery from leucopenia.

### Experimental Example 2

Effects On Activity of NK Cell

According to the experiment reports of the Military Medical Science Academy, China, the culture of the present invention was made into injection preparations of 500μ in each ml. Experimental tumors were s180 sarcoma, Lewis lung cancer, and U14 cervical carcinoma. Experiments were made on Kunming-species mice and C57BL/6 mice with the methods well known by the person skilled in the arts to measure activity of NK cells and lymphocyte transformation. The experiment results are as follows:

The NK cell activity was increased two days after one time of injection of the culture, reached the summit four days later, and restored to the level before the injection six days later. Its tumor suppression rate exceeded 90%.

The variation of one time dosage for S180 sarcoma is: the dosage of 200˜1000μ/mouse could slightly increase the NK cell activity, 1200˜1500μ/mouse could obviously increase the NK cell activity. For the test of Lewis lung cancer and U14 cervical carcinoma, the dosage of 32μ/time per day could evidently increases NK cell activity after continuous nine days' dosages (p<0.05).

Therefore, one time or repeated dosages of the culture can promote the NK cell activity of normal mice and tumor bearing mice.

### Experimental Example 3

Effects On Lymphocyte Transformation Rate

According to the experiment reports of the Military Medical Science Academy, China, one time dosage of 32.5μ applied to 10 normal mice could slightly increases their lymphocyte transformation rate. The effect became evident four days later and it restored to normal 6 days later.

Lymphocyte transformation rate of mice having tumor could be increased slightly after 9 days of continuous abdominal cavity injection of the culture obtained from example 1 with the dosage of 32.5μ. When mice had large dosage (for example: 1,000 or 1,500μ) once, their lymphocyte transformation rate could be increased evidently.

### Experimental Example 4

Inhibiting Effect On Growth Of Tumor Cells

According to the experiment reports of the Military Medical Science Academy, China, ascites was gotten from a S180 mouse having tumor cells. It was diluted and calculating the expected tumor cell number, and adding different quantities of the culture obtained in the example 1. The liquids were mixed up and subcutaneous inoculation was carried out immediately for each mouse with the dosage of 0.2 ml. The mice were killed 10 days later to get their tumors. The control group accepts physiological saline solution of the equivalent quantity instead of culture to be tested while the other conditions are kept the same as those of the experimental group. The results showed that the dosage of 50μ/mouse had evident inhibition effect S180's tumor growth. Compared with the control group, the average tumor inhibition rate was 38.3±20.9%. The inhibition effect was in direct proportion to the dosage. When the dosage is increased to 200˜1500μ, the inhibition rate could reach 91% maximum.

### Experimental Example 5

Therapeutic Effect on S180 Tumor

According to the experiment reports of the Military Medical Science Academy, China, 86 mice were divided into four groups, with a group of 23 mice as the control group and each of the other groups consisting of 21 mice. Subcutaneous injection of S180 ascites tumor cells was carried out for the mice in the experimental group. Then present culture of the invention obtained in example 2 was injected into the bodies of the mice 24 hours later, and kept on injecting once a day for 9 continuous days. The mice were killed on 10 days later to get their tumors and followed by weighting them. Physiological saline solution was used for the mice of the control group. The experimental results showed that the inhabiting rates of the dosages of 50, 100, and 150μ/mouse were respectively 25%, 30% and 37%.

Besides, it also displayed slight inhabiting effect to inject the culture of the present invention with the dosages of 50, 100 and 150μ/mouse once a day for 9 continuous days staring from the time 24 hours after subcutaneous inoculation of tumor.

### Experimental Example 6

Clinical Observation Results

With massive clinical experiments performed by China-Japan Friendship Hospital, Beijing, and Qingdao People's Hospital, Shenyang City's 5^{th} Hospital, Bengbu Medical College Affiliated Hospital, Dalian Medical College Affiliated Hospital and Shanghai Tongren Hospital, including radiotherapy and chemotherapy comprehensive experiments, and the experiments on the culture's effects on human bodies, it was proved that the invented culture had the capabilities to increase the amount of leucocyte after leucocyte decreased in radiotherapy and chemotherapy. Besides, the invented culture could also prevent leucocyte from decreasing when it was used during the radiotherapy and chemotherapy periods.

And the culture also had the effects to alleviate the side effects of radiotherapy and chemotherapy (such as suppression of bone marrow, the intestines and stomach responses, inappetency, loss of weight and the like).

According to the present invention, said culture is safe for animal including human. Some of the patients taking part in the experiments ran a fever of about 38° C. in the first 1˜3 day when the experiments started, and turned better quickly with treatment. Most of the participators could withstand it and recovered within one day automatically or with minor treatment.

Generally, the comprehensive curative effects are determined as follows: notable effective rate: 25.93%, effective rate: 55.09%, improvement rate: 15.74% and no effect: 3.24%, so the total effective rate (the notable effective rate plus the effective rate) is 81.02%.

We claim:

1. A process of producing a Staphylococcus Aureus culture including the steps of:
(1) putting the raw material of pig heart into 1˜2 times of water, and shattering and filtrating it to obtain the first filtrate and the first filter residue;
(2) putting 1˜2 times of water into the first filter residue and immersing and filtrating the same at 90˜95° C. to obtain the second filtrate and the second filter residue; mixing the first and second filtrate to obtain the third filtrate;
(3) putting 0.025˜0.5% of peptone and 0.3˜0.9% sodium chloride into the third filtrate based on the total liquid weight,, and adding 0.5% active carbon, adjusting its pH value to about 7.2 to obtain a culture medium;
(4) reactivating Staphylococcus aures, and preparing seed liquid after bacteria number is increased, inoculating the a culture medium with a rate of 0.02%˜0.2% ml % after bacteria concentration reaches 10⁵-10⁷;
(5) fermenting inoculated medium at about 35° C. for 15˜20 hours to get a culture;
(6) removing the bacteria , filtrating said culture, and adjusting its osmotic pressure to make it isotonic, and pH value to 6.8˜7.4, so as to obtain the final product.

2. A strain of Staphylococcus aures having been deposited in China CGMCC on Sep. 14, 2000, with a deposit No. CGMCC0485.

3. The process as claimed in claim 1, wherein the culture medium includes 0.5˜10 ml of pig heart filtrate, 0.025˜0.5% of peptone and 0.3˜0.9% sodium chloride based on the total medium weight.

4. A Staphylococcus aures culture produced by the process of claim 1.

5. The use of the A Staphylococcus aures culture as claimed in claim 4 in the preparation of a medicament for anti-leucopenia caused by chemotherapy.

6. The use of the A Staphylococcus aures culture as claimed in claim 4 in the preparation of a medicament for the treatment of tumor.
